(19) United States
(12) Patent Application Publication
(10) Pub. No.: US 2021/0015659 A1
(43) Pub. Date: Jan. 21, 2021

(54) Therapeutic eye mask

(71) Applicants: Kam Cham Lau, San Francisco, CA (US); Jin Zhang, Beijing (CN)

(72) Inventors: Kam Cham Lau, San Francisco, CA (US); Jin Zhang, Beijing (CN)

(21) Appl. No.: 16/602,473

(22) Filed: Oct. 15, 2019

(30) Foreign Application Priority Data
Jul. 16, 2019 (CN) 201921127519.0

(51) Int. Cl. A61F 7/00; A61F 9/04

(52) U.S. Cl. CPC A61F 7/007; A61F 9/04; A61F 2210/0076; A61F 2007/0077; A61F 2007/0004

(57) ABSTRACT

The therapeutic eye mask includes a multi-layer composite main body having a heater plate and a plurality of vibrators mounted therein. The heater plate is located over the wear's eyes when the mask is worn. The vibrators are located in upper locations relative to the heater plate. The heater plate and the vibrators are operated by a pulse control circuit and a timer which are operative by an electrical controller connected to an electrical supply with a USB connector. A space cloth plate and a plurality of magnet disks are provided in the main body. The electrical controller has a first switch operative to connect the eye mask to the electrical supply and a second switch is operative to facilitate the user in choosing selective massage motions to provide effective therapeutic operation.

### BACKGROUND OF THE INVENTION

### 1. Field of the Invention

This invention relates to a therapeutic eye mask and particularly an eye mask for massaging and promoting blood circulation around the eyes to provide health benefits and cure of eye disease such as dry eyes.

### 2. Background Art

Nowadays, with the widely use of computer, television, and mobile telephone provided with an electronic video display it has caused increasing stresses to the human vision, and it can lead to a variety of eye sicknesses such as dry eyes.

A therapeutic eye mask for easing vision stresses and curing dry eyes is shown in Chinese Patent No. CN208372009U issued on Jan. 15, 2019 to the joint applicant of the present application. The mask is provided with a vibrator and a heat plate which applies heat and massage action to the face around the eyes for increasing blood circulation. The heat plate of the device is first activated to apply heat to warm the blood veins in the eyes to increase the blood flow around the eyes and then followed by activating a massage vibrator to generate massaging frequency of 9700 to 10300 Hz with an energy of 0.001 watt to massage the muscles around the eyes such that in combination the heat and the massage action provide stress release for the eyes and it is helpful in effectively curing eye diseases such as dry eyes. However, the massaging function of such eye mask is restricted to a fixed pattern. Prolong use of such single massage pattern or motion would reduce its effectiveness due to molecular muscular fatigue induced in the eye muscle exposed to only same single massage pattern. Furthermore, different massage patterns must be required for different persons such that the fixed single massage pattern may provide only limited effectiveness to some people. Still furthermore, known eye masks are commonly made of relatively heavy material such that the excessive weight of the mask can increase stress to the eye muscles of the wearer. Some masks also are made of materials which are conducive to bacterial growth, such that use of such eye masks would increase the chances of catching various eye infections.

### SUMMARY OF THE INVENTION

It is a principal object of the present invention to provide a therapeutic eye mask capable of providing variable massaging patterns to the eyes for effective therapeutic purposes.

Briefly for overcoming the above drawbacks of the known eye mask, the present eye mask is provided with a heater plate and a plurality of vibrators with the operation of which controlled with a pulse control circuit and a timer. The operation electrical supply to the pulse control circuit and the timer is controlled with a manually operable switch and the pattern of massage action can be selected with a second manually operable override switch. Electrical supply is provided to the control with a USB connector. Magnet disks are provided within the mask to apply additional magnetic force to the eyes and a spacing fabric is provided on the rear underside of the mask adjacent to the face of the wearer .

The device is characterized with the provision of a manual switch which is operable for activating the device with the electrical supply to immediately turning on the heater plate to generate the warming heat to increase the blood circulation in the eyes. The pulse control circuit will automatically turn on the vibrators to provide the massage actions after the heater plate has been turned on for 6 minutes. The combination of heat and massage actions will then operate for a total period of 15 minutes to provide the curing operation.

Another object of the present invention is to provide a therapeutic eye mask having an electrical control provided with indicating lights showing the operation of the heater plate and the massage vibrators.

Another object of the present invention is to provide a therapeutic eye mask in which the heater plate provides a stable warming temperature of between 43 to 46° C.

Still another object of the present invention is to provide a therapeutic eye mask having a composite multi-layer main body with a sponge material sandwiched between a front layer and a rear layer. A heater plate, a plurality of vibrators, and a plurality of magnet disks are located in the layer of sponge material, and the magnet disks are located in locations relatively below the locations of the vibrators to provide additional magnetic field to the meridian of the eyes to relieve stresses of the eyes.

Another object of the present invention is to provide a therapeutic eye mask having an adjustable mounting belt for mounting the mask to the head of the wearer. The belt is mounted to two buckles located at the lateral sides of the frame of the mask adjacent to the layer of sponge material

Another object of the present invention is to provide a therapeutic eye mask with a multi-layer composite main body having a rear layer made of a smooth resilient fabric material to increase the comfort for the wearer.

Another object of the present invention is to provide a therapeutic eye mask wherein the front layer of the main body is made of a resilient plastic material spray-treated with silicon for easy cleaning and bacterial resistance.

Another object of the present invention is to provide a therapeutic eye mask having a one time use disposable cloth plate removably mounted over underside of the main body.

Another object of the present invention is to provide a therapeutic eye mask having two elastic mounting cords provide at the main body for removably mounting the disposable cloth plate to the rear underside of the main body.

Yet another object of the present invention is to provide a therapeutic eye mask which obviates the drawbacks of the known eye masks in that the operation of the vibrators is controlled by the pulse control circuit to provide a plurality of selective massaging motions which can avoid the fatigue effect of the eye muscles to a fixed massaging motion. The user can freely select the desirable massaging motion by experimentation with simple operation of the override switch. Furthermore, the rear layer of the main body of the eye mask of the present invention is made of a smooth resilient fabric material, and the front layer is made of silicon spray-treated smooth soft plastic material to greatly reduce the contamination by bacteria such that it is a bacterial free healthy product.

### BRIEF DESCRIPTION OF THE DRAWINGS

Other objects and advantages of the present invention will become apparent from the following detailed description of the preferred embodiments thereof in connection with the accompanying drawings, in which

FIG. 1 is a perspective rear elevation view of the eye mask of the present invention having an adjustable belt for mounting the mask to the head of a user.

FIG. 2 is a cross section side elevation view along section line II-II of the multi-layer main body of the mask of FIG. 1.

FIG. 3 is a perspective front elevation of the disposable fabric plate for use with the eye mask

### DETAILED DESCRIPTION OF THE PREFERRED EMBODIMENTS

With reference to the drawings, the eye mask of the present invention has a generally oval shape frame 1 having a W-shaped lower edge and dimensions for comfortably covering over the face area of both eyes of a person. A main body 2 shaped as the frame of the mask is mounted within the frame 1. The main body 2 is a composite multi-layer plate having an front layer 3 made of a flexible plastic material spray-treated with silicon to have a smooth texture such that it is bacterial resistant yet can be easily cleaned. The rear layer 4 of the main body 2 is made of a resilient fabric which is bacteria resistant, smooth in feel, such that the mask can be worn comfortably over the user's face with the rear layer 4 adjacent to the user's face without causing any stress to the eyes. A resilient layer 5 of a resilient material such as natural or synthetic sponge is sandwiched between the front layer 3 and the rear layer 4 of the main body 2.

A heater plate 6 is also mounted between the resilient layer 5 and the rear layer 4 in the main body 2. The main body 2 with the heater plate 6 sandwiched therein covers flatly over both eyes of the wearer when the mask is worn. The heater plate 6 can be electrically operated to provide a stable warming temperature of 43 to 46° C.

Two vibrators 7 and 8 are provided in the main body 2 and are located just above the eyes of the mask user when the mask is worn. The distance between the two vibrators 7 and 8 is preferably between 62 to 66 mm apart from one another. The vibrators 7 and 8 provide vibration frequency of massage motion of between 7000 to 13000 Hz with an output energy of 0.001 watt.

A plurality of magnet disks 9, 10, and 11 are mounted within the main body 2 to apply additional magnetic field curing to the eyes. The central magnet disk 10 is located at the center of the main body 2, and the other two side magnet disks 9 and 11 are located at 5.6 mm spaced from the left and right sides of the main body 2 respectively. The magnet disks 9, 10 and 11 are made of a strong magnetic material such as neodymium (Nd₂Fe₁₄B), circular in shape and having a diameter of 5 mm, and thickness of 3 mm. The magnet disks 9, 10, and 11 are additionally fixedly mounted in place with adhesive within the main body 2.

An adjustable belt 12 is provided for the mask for mounting the mask to the head of the wearer. The two ends of the adjustable belt 12 are inserted and looped through two buckles 13 and 14 provided at the left and right sides of the frame 1. The adjustable belt 12 is operable to mount the mask over the head of the wearer with the main body 2 of the mask resting comfortably and intimately over the eyes of the wearer.

Electrical current is supplied to the heater plate 6 and the vibrators 7 and 8 through an electrical circuit in a control unit 15 which can be connected to a power supply through a connector 16 such as a USB connector.

The electrical circuit includes a pulse control circuit and a timer. The pulse control circuit operates to activate the vibrators to provide a plurality of selective massage motions, and the timer controls the time period the massage motions as well as the heater are being activated. Typically, the heater is first activated for a period of 6 minutes before the vibrators are activated, then the combination of both heat and massaging motions will be operated for a total time period of 15 minutes to provide the required therapeutic effect. A first depressible on/off button switch 17 with an indicator light is provided at the control unit 15 for the user to manually turning the power supply to the mash on and off; and a second depressible on/off button override switch 18 with an indicator light is provided at the control unit 15 for the user to manually activating the pulse control circuit. The first depressible on/off switch 17 and the second depressible on/off switch 18 may be different in size and the indicator lights of different colors, for example, red for the first on/off switch 17 and a second color for the second on/off switch 18, to facilitate the user in identifying these switches of the different functions.

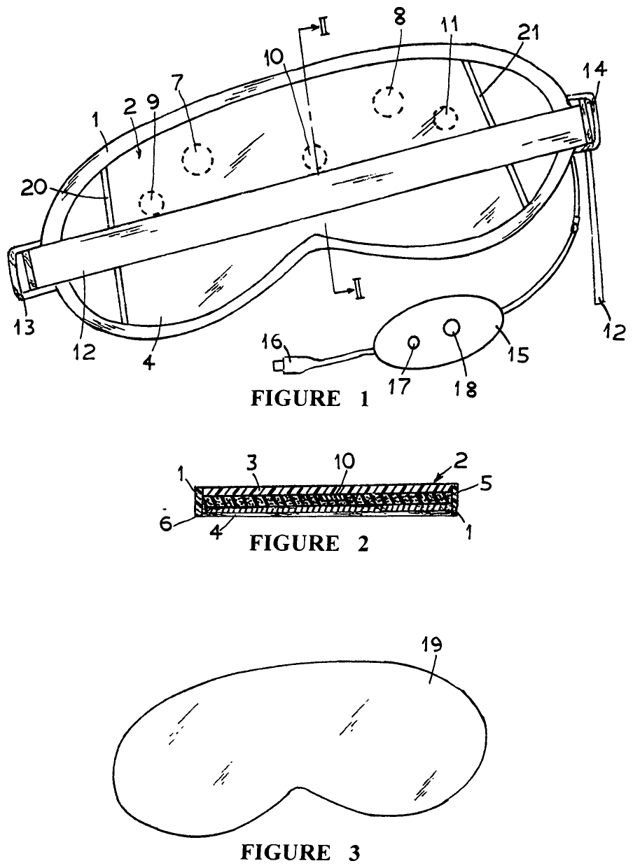

A disposable cover sheet 19 as best shown in FIG. 3 having a shape and dimensions similar to the main body 2 may be provided for covering over the rear surface 4 of the main body 2. The disposable cover sheet 19 may be removably mounted over the rear layer of the main body 2 with two elastic cords 20 and 21 extending transversely to the two side portions of the frame 1 as best shown in FIG. 1. The disposable cover sheet 19 may be slipped under the elastic cords 20 and 21 to be held removably in place over the rear layer 4 of the mask prior to mounting the mask over the face of the wearer so as to prevent contamination of the mask in contact with the face of the wearer.

In operation, a disposable cover sheet 19 is first mounted to the underside over the rear layer of the mask, and the mask is then worn over the head of the wearer with the adjustable belt suitably adjusted such that the mask covers comfortably over the eyes of the wearer. The USB connector 16 is then connected to a power supply of 3V dc of 80 mA. The first on/off switch 17 is then depressed for about 3 seconds. The red indicator of the first on/off switch 17 will be lighted to indicate that power is supplied to the mask and the heater plate 6 is activated. The temperature of the heater plate 6 will eventually reach a stable temperature of 43 to 46° C. After another 4 minutes the second indicator light will be lighted to indicate the vibrators 9, 10, and 11 are being activated. Then the combination of heat and massage motions will operate for a total period of 15 minutes, at the end of the operation both indicator lights will automatically turn off to show the termination of the therapeutic function. The entire operation is simple and automatic.

Different selective massage motions may be chosen by the user during operation by depressing the override switch 18 until a selective desirable massage motion is provided by the vibrators 9, 10, and 11.

What we claim is:

1. A therapeutic eye mask comprising:
a composite multi-layer main body mounted inside a flexible plastic frame, said main body being mountable over eyes of the face of a user of said mask; said main body having a fabric rear layer;
a plurality of magnet disks mounted in said main body;
a heater plate and a plurality of vibrators mounted inside said main body;
an electrical controller electrically connected to said heater plate and said vibrators;
said electrical controller having a pulse control circuit and a timer; and a USB connector for connecting said electrical controller to an electrical supply;
a first switch provided on said electrical controller, said first switch being operative for activating said heater plate and said vibrators;
a second switch provided on said electrical controller, said second switch being operative for selection of a plurality of massage motions by said vibrators.

2. A therapeutic eye mask according to claim 1 wherein said first switch is operative to activate said heater plate and said vibrators are activated by said pulse control circuit after said heater plate has been activated, and said heater plate and said vibrators being operative in combination for a total period of 15 minutes.

3. A therapeutic eye mask according to claim 2 including a first indicating light operative to indicate said heater plate has been activated, and a second indicating light operative to indicate said vibrators have been activated.

4. A therapeutic eye mask according to claim 3 wherein said heater plate reaches a stable warming temperature of 43 to 46° C. in 2 minutes after being activated.

5. A therapeutic eye mask according to claim 1 including a inner layer of resilient sponge material sandwiched between a front layer and a rear layer of said composite multi-layer main body; and said heater plate, vibrators, magnet disks being located within said inner layer.

6. A therapeutic eye mask according to claim 1 including an adjustable belt mounted to two buckles located at two ends of said frame, said belt being adjustable for mounting said eye mask comfortably on the face of a wearer over the wearer's eyes.

7. A therapeutic eye mask according to claim 6 wherein said composite multi-layer main body has a rear layer made of a smooth feel, resilient fabric material.

8. A therapeutic eye mask according to claim 7 wherein said composite multi-layer main body has a front layer made of a silicon spray-treated plastic material.

9. A therapeutic eye mask according to claim 8 including a one time use disposable cloth plate mountable removably over said rear layer of said main body.

10. A therapeutic eye mask according to claim 9 including two elastic cords located transversely over two side portions of said rear layer for removably mounted said disposable cloth plate on said mask.
